(19) United States
(12) Patent Application Publication
(10) Pub. No.: US 2015/0038878 A1
(43) Pub. Date: Feb. 5, 2015

(54) DEVICE AND METHOD FOR DETERMINING AND DISPLAYING FORCES

(71) Applicant: OTTO BOCK HEALTHCARE GMBH, Duderstadt (DE)

(72) Inventor: Martin Pusch, Duderstadt (DE)

(73) Assignee: OTTO BOCK HEALTHCARE GMBH, Duderstadt (DE)

(21) Appl. No.: 14/379,465

(22) PCT Filed: Feb. 15, 2013
(86) PCT No.: PCT/EP2013/000452

§ 371 Date: Aug. 18, 2014

(30) Foreign Application Priority Data
Feb. 17, 2012 (DE) 102012003033.9

(51) Int. Cl. A61B 5/103; A61B 5/00

(52) U.S. Cl. CPC A61B 5/1036; A61B 5/4851; A61B 5/742; A61B 5/7278
USPC 600/587

(57) ABSTRACT

The invention relates to a method for determining and displaying horizontal and vertical forces which act on a person when standing, in which method the person stands on two separate force measurement plates coupled to each other, wherein each foot is positioned on a respective force measurement plate, and the respective support point on the force measurement plate is determined via the force measurement plates, wherein a resulting force vector (FRR, FRL) is calculated from the respective support points of the two force measurement plates, from a known point of gravity of the body and from a vertical force distribution of the two force measurement plates relative to each other, and is displayed using a display device.

The invention relates to a method for determining and displaying forces acting on a person while standing, in which the person stands on two separate, intercoupled force measurement plates, one foot each being positioned on a force measurement plate, and the respective support point on the force measurement plate being determined via the force measurement plates. The invention likewise relates to a device for carrying out such a method.

EP 663 181 A2 describes a display system for measuring the human body, and a corresponding method in which a patient stands on a measurement plate and is provided with a vertical centroid line in the form of a laser beam. The light beam moves in one or the other direction depending on the centroid displacement. Said display system can be used to achieve a more precise adjustment of a prosthesis setup, that is to say the assignment of the individual prosthesis components for prostheses of the lower extremity, so that it is possible to attain improved functionality and greater comfort for the prosthesis wearer.

WO 2002/059 554 A2 discloses a method and a device for monitoring forces, in particular forces that are exerted by a worker on other objects. For this purpose, the worker is located on a force measurement plate which acquires the forces in three spatial orientations. The forces acquired are displayed on any desired display device.

U.S. Pat. No. 4,598,717 A1 relates to the acquisition of static and dynamic body loads with the aid of pressure measurement plates so as to determine horizontal forces.

DE 10 2009 003 487 A1 relates to a device having two separate force measurement plates, permanently assigned to one another, and three display devices which display the respective centroid line of the force measurement plate on the body of the person standing on the force measurement plates. The light lines are projected onto the body. In addition to displaying the individual centroid positions on projection planes in the sagittal plane and frontal plane, it is also possible to display an overall centroid. Also provided are manually adjustable display devices for displaying desired positions or reference planes. The distance between individual centroid positions, or a desired position and a centroid position can likewise be displayed.

DE 10 2006 021 788 A1 relates to a device for determining and displaying a horizontal force component with the aid of a force measurement plate, in the case of which the display is performed on the body image. The display can be performed via a beamer.

It is an object of the present invention to provide a method and a device in which a horizontal force component acting on a patient can be determined and displayed cost—effectively and reliably.

Said object is achieved according to the invention with a method having the features of the main claim and a device having the features of the coordinate claim. Advantageous refinements and developments of the invention are disclosed in the subclaims, the description and the figures.

The method for determining and displaying horizontal and vertical forces acting on a person while standing, in which the person stands on two separate, intercoupled force measurement plates, one foot each being positioned on a force measurement plate, and the respective support point or the centroid position on the force measurement plate being determined via the force measurement plates, provides that a resulting force vector is calculated from the respective support points of the two force measurement plates, from a known body centroid height and from a vertical force distribution of the two force measurement plates relative to one another and is displayed with the aid of a display device. Thus, it is provided here to calculate, a separate, simultaneous examination of the support points and of the centroid positions under the two feet, a horizontal force component which is displayed with the adoption of the body centroid height with the aid of a display device, for example a display screen or a projector. The display can be performed directly on the body of the person or by an obliquely represented force vector which is superposed or displayed in a recorded image shown on the display screen or the like. A reliable display of the tilt angle of the resulting force vector preceding from the body centroid height is possible even given plates with unequal vertical loads. The individual support points or centroids can be acquired cost—effectively and reliably by simple pressure sensors. The sensors are effective in particular only in a vertical direction, similar to scales. The persons standing on the force measurement plates can be recorded via a camera device, and the recorded image can be displayed with a display device. The display of the resulting force vector can be performed on the grounds of a determined horizontal force component and of a vertical force component with the origin at the body centroid height, it being possible to perform the representation in a recorded image or film, or directly on the person and the background located behind.

The vertical force component, with respect to the respective resulting support point, can be represented separately for each leg to obtain a visual impression of the force introduction point of the vertical force. A precise analysis of the weight distribution can be undertaken for each foot owing to the separate representation.

An overall centroid can be determined from the support point data, that is to say the position of the respective force measurement plate, often the centroid position data of the force measurement plates, and the resulting force vector can be calculated and displayed for each force measurement plate. The representation or display of the resulting force vector can be performed, with the origin at the overall centroid, in the display image or on the person. The overall centroid is preferably displayed above a centroid line which is superposed in the display image of the person or is projected onto the person. It is possible in this way to display the action of the horizontal force component very clearly via the oblique, resulting force vector.

In order also to be able to undertake a quantitatively relative display of the forces acting, it is provided that before the resulting force vector is displayed sensors or markers at at least one force measurement plate are used for autocalibration of the display device relative to the force measurement plate. The display of the force vectors can therefore be performed true to scale in the image. The autocalibration can be performed via a plurality of markers or sensors, in particular photosensitive sensors, which are arranged at at least one force measurement plate, preferably at all the corners of the force measurement plates. A measurement field in which the force measurement plates and, if appropriate, also the person located on the force measurement plates is scanned so that it is possible to determine the dimensions of the force measurement plates, their position in space and the location at which the resulting force vector is displayed, thus enabling a qualitatively and quantitatively correct display.

The autocalibration can be performed by arranging photosensitive sensors at measurement points of at least one force measurement plate, and carrying out the autocalibration by illuminating the entire measurement field and subsequently reducing the illuminated area and selectively activating the individual sensors. This can be done, for example, by arranging the image section of the camera device parallel to an edge at one of the force measurement plates, and by directing onto the sensors at the display device a light field, for example a beamer, which firstly illuminates all the sensors and is subsequently reduced for each sensor until only one sensor is still being illuminated. This is repeated for all the photosensitive sensors so that the individual sensors are individually approached successively, thus identifying the positioning between the force measurement plate and the display device together with the focal length and orientation of the beamer. The horizontal force measurement plate can be aligned in this case so that it lies at right angles at the lower image edge of the image of the display device. The image of the display device, for example of the beamer, is to be adjusted to the extent that a person on the plate is illuminated at least to the height of the body centroid. A vertical orientation of the image of the display device has an advantageous effect as regards the image brightness. A distance between the display device and the force measurement plate that is as large as possible reduces the parallex error.

For the purpose of autocalibration, if the measurement plate is of a rectangular design the photosensitive sensors can be arranged at its four corners and then be irradiated by the display device, for example by the beamer. The display device illuminates the entire image area such that all the photosensitive sensors detect the signal and transmit corresponding data to an evaluation device. Beginning from one direction, for example from above, the luminous area of the display device, designed as a blinking surface, for example, is reduced such that the lowermost cell is still just illuminated. The same operation is repeated from the four other directions, that is to say from below, from the left and from the right such that the exact position of said one sensor is detected. Said operation is repeated in the same way for the other sensors such that the positioning between the force measurement plate or between the force measurement plates and the display device can be determined together with the focal length and the orientation of the beamer. It is possible in this way to carry out autocalibration of the display device and of the displayed force sensors within the image.

It can be provided for the purpose of autocalibration that the display device projects patterns into the measurement field and adapts them such that they correspond to the sensors or markers at at least one force measurement plate, the sensors or markers thus being precisely illuminated or excited.

It can also be provided that the autocalibration is carried out by the display device as a function of patterns or coordinates in the measurement field. Patterns or specific points can be detected in the measurement field. The calibration can also be performed by the user employing a mouse cursor to approach and confirm specific patterns or calibration points.

It is possible to provide that the image of the display device, that is to say the resulting force vector, is projected onto the person standing on the force measurement plates, it being possible to record this in turn with the camera device. It is likewise possible for each individual force component of the force measurement plates to be displayed in the image, that is to say for each horizontal force component of the individual force measurement platforms to be separately displayed so that the horizontal force component exerted by each leg is displayed in the image, preferably as an oblique force vector of the resulting force. The resulting force vector can, in particular, be displayed in an image on a monitor such that the point of application, the orientation and the amplitude of the vector can be in the image positioned correctly and scaled.

It is advantageously possible for there to be arranged next to the force measurement plates light, preferably white surfaces which are suitable for projecting additional data in the image, it also being possible, optionally, for the projection to be performed onto the white surfaces themselves so that the person standing on the force measurement plates can obtain information during recording of the measurement values.

The body centroid height can be input manually or determined by detecting the horizontal force in the frontal plane or sagittal plane. The body centroid height can be determined during detection of the horizontal force in the frontal plane, and can also be used in the sagittal plane.

The force vector can be superposed on the image recorded by the camera device both in the frontal plane and in the sagittal plane by projecting the force vector into the image. The load exerted by the standing person in both planes is then visualized.

The device for carrying out a method as claimed in one of the preceding claims which has two force measurement plates that are provided with sensors for recording forces and are connected to an evaluation device via which the acquired measured variables are evaluated, provides that the force measurement plates are mounted decoupled in at least one horizontal force direction. For this purpose the force measurement plates are preferably mounted on rollers which are advantageously guided parallel to one another. The plates are held at one point on the frame relating to the horizontal force, this being possible with sufficient flexibility with the aid of soft silicone.

The sensors, in particular horizontal force measurement cells, can also be fitted below the force measurement plates. The measurement sensors can be designed as horizontal force measurement cells which are arranged underneath or alongside the respective force measurement plate. The horizontal force measurement cells are joined to the force measurement plates so that the bending of the force measurement plates under the load of the patient does not affect the result. This can be achieved, for example, by an articulated bearing or push rods.

In addition to the decoupling in different horizontal force directions, it is possible for the two force measurement plates to be mounted decoupled in two horizontal force directions, for example by means of the sliding or rolling bearing arranged crosswise. Alternatively, the force measurement plate can be mounted on an elastomeric intermediate layer, or on bearings of lenticular cross section. Dimensionally stable one- or two-dimensionally curved elements or materials which do not, or scarcely, transmit shear forces are suitable in principle for bearing purposes so that no substantial resistance is offered to a displacement in a horizontal direction in the relevant range of movement.

One development of the invention provides that the sensors can be designed as rolling elements on which the force measurement plate is mounted. It is thereby possible to enable the force measurement plates to be mounted directly on the sensors or vertical force cells so that there is no longer a need to provide separate bearings in order to effect a decoupling of the force measurement plates in at least one horizontal direction. The sensors can be designed as spheres or combination of spherical parts, for example by configuring the upper and lower connection surfaces of the sensors as spherical surfaces which are outwardly arched in order to fulfill a roller bearing function. Barrel-shaped configurations or cylindrically or partially cylindrical configurations can also be provided in addition to a spherical configuration.

Exemplary embodiments of the invention are explained in more detail below with the aid of the attached figures in which:

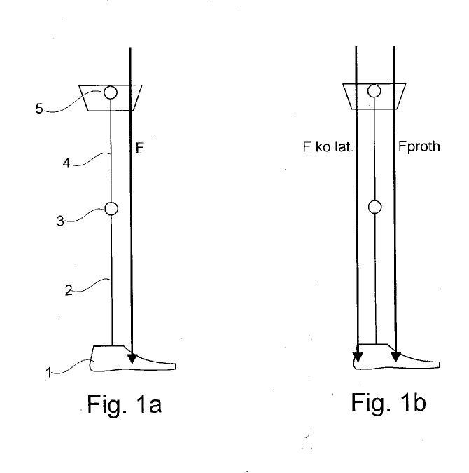

FIGS. 1a and 1b show different force introduction points in the sagittal plane;

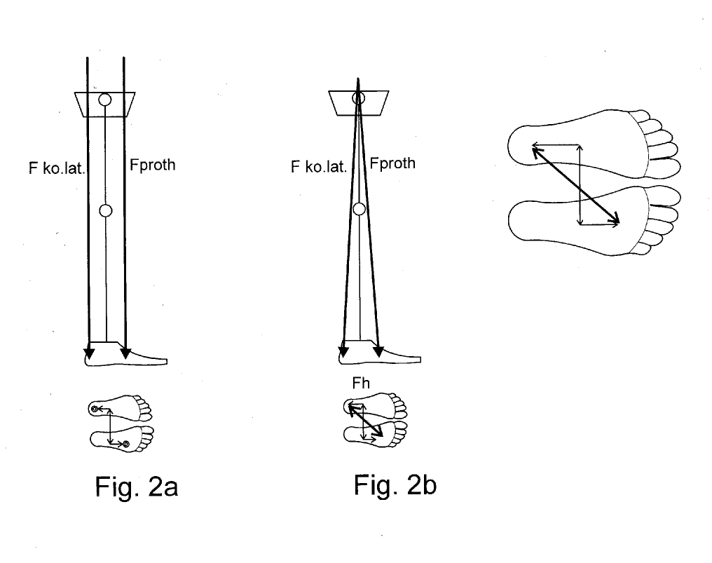

FIGS. 2 and 2b show force profiles in the sagittal and transversal planes with and without a horizontal force component;

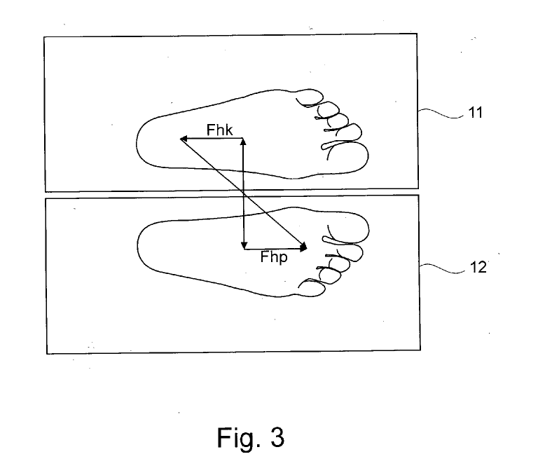

FIG. 3 shows an illustration of the influence of horizontal forces in the case of mutually corresponding horizontal force influences;

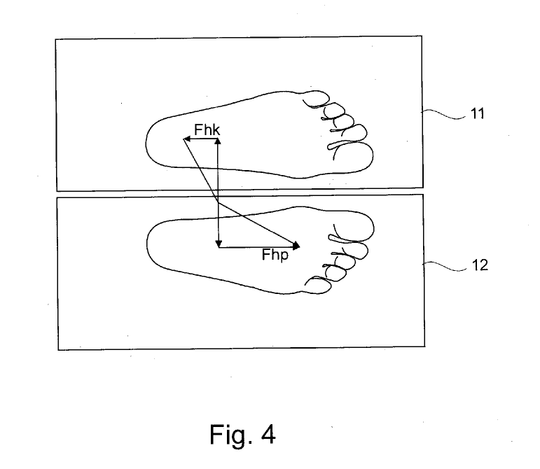

FIG. 4 shows an illustration in accordance with FIG. 3 with different horizontal force influences;

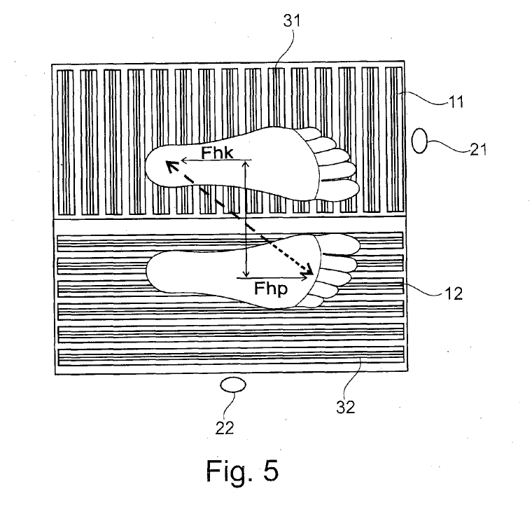

FIG. 5 shows a force measurement plate structure with decoupling in a horizontal force direction;

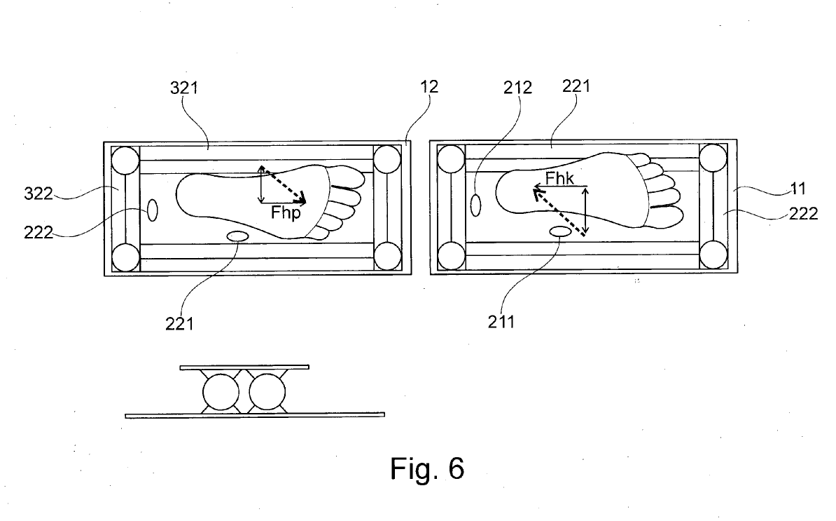

FIG. 6 shows a dynamically stacked force measurement plate structure;

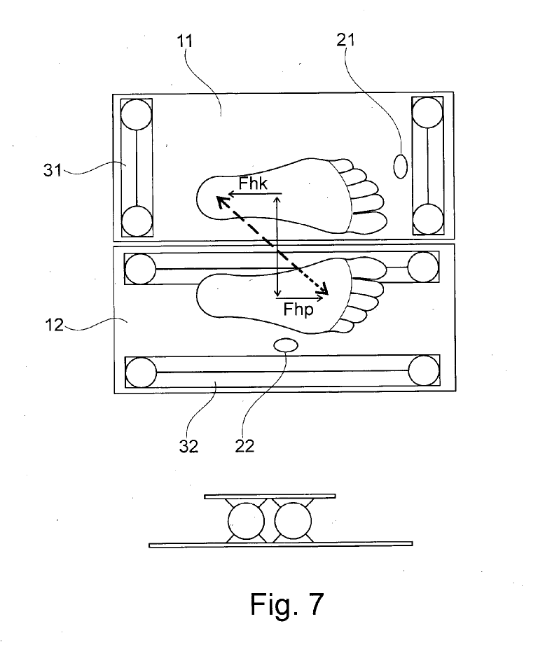

FIG. 7 shows an arrangement of horizontal force measurement cells under the force measurement plates;

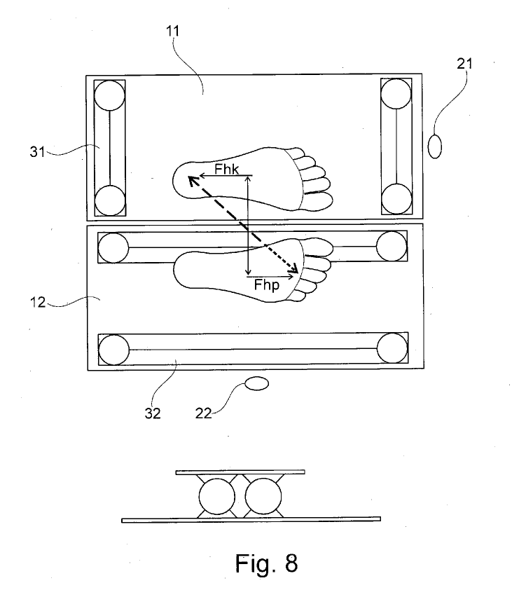

FIG. 8 shows an arrangement of force measurement cells alongside the force measurement plates;

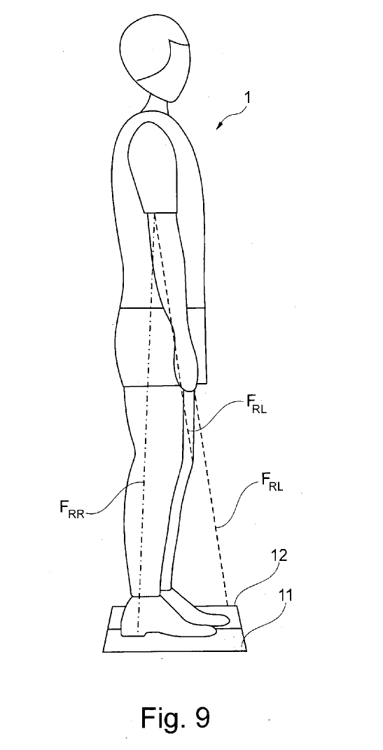

FIG. 9 shows an illustration of a force vector projection of a person;

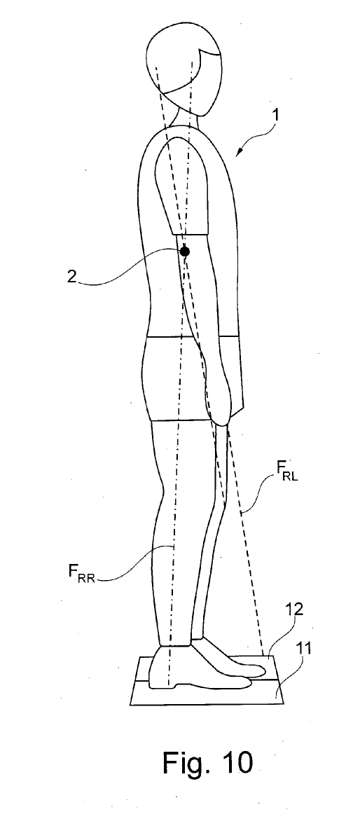

FIG. 10 shows an illustration in accordance with FIG. 9 through a known body centroid;

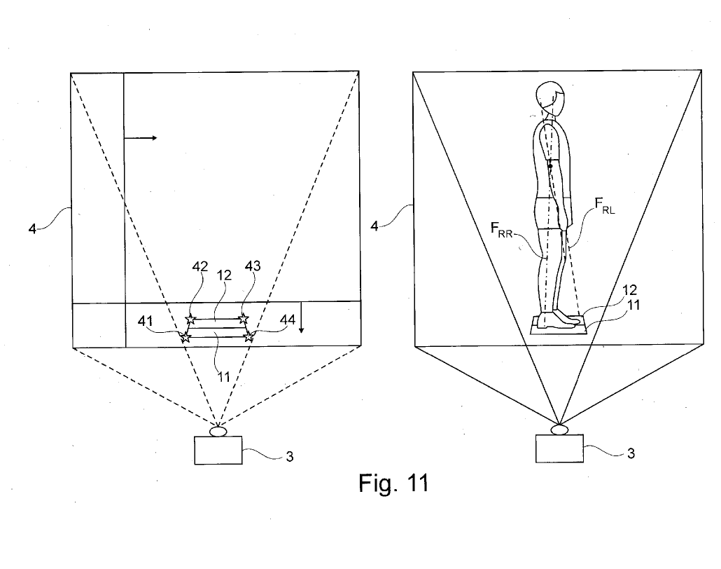

FIG. 11 shows a schematic illustration of an autocalibration;

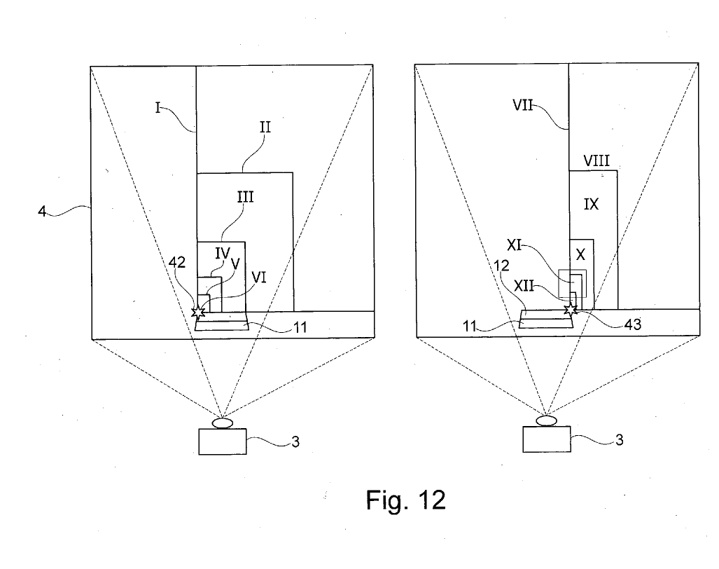

FIG. 12 shows a variant of autocalibration;

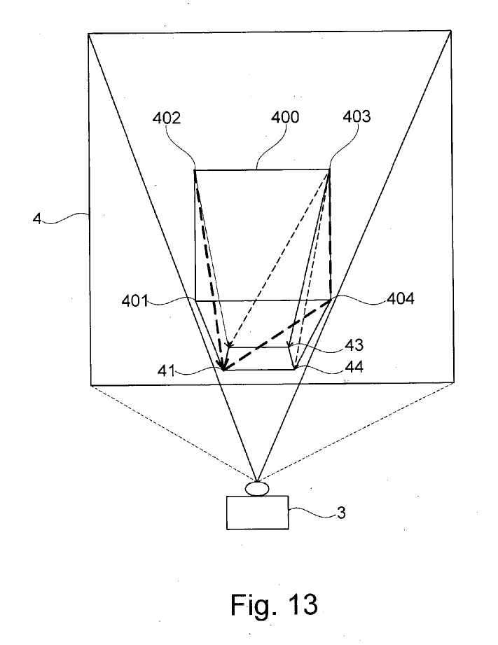

FIG. 13 shows an autocalibration with adaptation of a pattern through a sensor surface; and

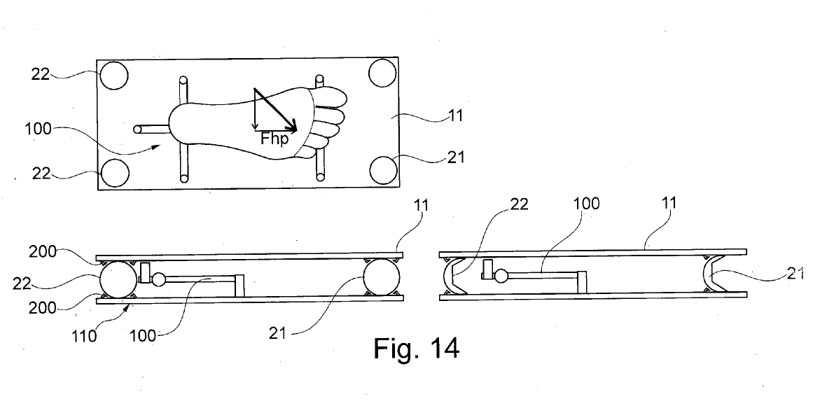

FIG. 14 shows a variant of the force measurement cells.

FIG. 1a is a schematic illustration of a prosthetic leg 1 having a lower leg part 2, a prosthetic knee joint 3, a thigh part 4 and a hip 5. An analogous structure also results in the case of a healthy leg. During normal standing, the force vector F of the ground reaction force is located in front of the knee joint 3, thus ensuring that standing with a prosthesis is reliable.

The force vectors Fₚᵣₒₜₕ for a prosthetic leg and F_{ko.lat} for the contralateral and healthy leg are illustrated in FIG. 1b. It is usual to assume uncomfortable standing when the difference between the two force vectors is greater than 10 mm.

In addition to the illustration of the two force vectors F_{ko.lat} and Fₚᵣₒₜₕ in the sagittal plane, FIG. 2a also illustrates a plan view in the transverse plane. The force introduction points of the force vectors are arranged in FIG. 2a on different sides of the vertical plane through the connecting line of the two knee joints: here, the force introduction point on the contralateral, healthy side is arranged behind the connecting plane in the normal direction of motion, while that of the prosthetic leg lies in front of the plane. No horizontal force component is present.

FIG. 2b illustrates the situation with a horizontal force component, in the case of which the horizontal force on the contralateral side is oriented rearward, while that on the prosthetic side is oriented forward.

In this case, the left foot is loaded rearward, for example in order to bring the hip forward, the right foot works counter thereto.

FIG. 3 illustrates the standing situation in accordance with FIG. 2b in a transverse plane, the two feet being respectively arranged on a force measurement plate 11, 12. Arranged under the two force measurement plates are force sensors which acquire the centroid position on each force measurement plate 11, 12. It is usual for only vertical force sensors to be arranged at the four corners in the force measurement plate so that the centroid position on each individual plate can be determined by a simple evaluation of the individual measurement signals of each force measurement plate. The two force measurement plates 11, 12 are mounted separately from one another so that they do not influence one another. The force measurement plates are intercoupled for measurement such that it is possible to determine an overall centroid by evaluating all the force sensors under the force measurement plates 11, 12. The respective horizontal force component Fₕₖ, Fₖₚ on the contralateral side or the prosthetic side can be determined from said overall centroid and the individual centroid of each force measurement plate 11, 12. If the vertical force component on the contralateral side Fᵥₖ is equal to the vertical force component on the prosthetic side Fᵥₚ, the horizontal force components Fₕₖ, Fₖₚ on the contralateral and prosthetic side, are also equal, and so the measured force corresponds to the reaction force.

Another load situation is illustrated in FIG. 4: the horizontal force component on the contralateral side Fₕₖ is smaller than the horizontal force component on the Fₕₚ on the prosthetic side, and the measured force corresponds to the reaction force when the vertical forces are different, specifically when the vertical force on the contralateral side Fᵥₖ is greater than the vertical force on the prosthetic side Fᵥₚ.

FIG. 5 illustrates a variant of the invention in which the force measurement plates 11, 12 are mounted on a series of rollers 31 which decouple horizontal forces in the respective direction. In the exemplary embodiment illustrated, the left force measurement plate 11 is mounted on rollers 31 whose axes of rotation is oriented essentially perpendicular to the longitudinal direction of the foot, while the right force measurement plate 12 has rollers 32 mounted oriented perendicular thereto. Horizontal force sensors 21, 22, which acquire horizontal forces effective in the operating direction of the rollers, are arranged laterally alongside the force measurement plates 11, 12. The friction of the rollers on the force measurement plates 11, 12 prevents the rotation of the force measurement plate about the vertical axis so that the respective force measurement plates 11, 12 can be displaced only in precisely one operating direction. In addition, or as an alternative to making an evaluation by determining the centroids, a horizontal force component is achieved by separate horizontal force measurement cells 21, 22.

FIG. 6 illustrates a variant of FIG. 5 in which the force measurement plates 11, 12 are horizontally forced-decoupled in both directions, the horizontal force measurement cells 211, 212, 221, 222 being arranged underneath the force measurement plates 11, 12 in the roller stacks. The horizontal force measurement cells 211, 212, 221, 222 are advantageously coupled to the force measurement plates 11 such that the result of bending of the individual plate has no influence on the measurement result, the horizontal force measurement cells 211, 212, 221, 222 being coupled advantageously to the force measurement plates 11, 12 via push rods or articulated devices.

FIG. 7 illustrates a further variant of the invention in which only one horizontal force measurement cell is provided in the respective operating direction; the horizontal force measurement cells in FIG. 6 are effective in both planes.

A further variant is illustrated in FIG. 8, where the structure of the force measurement plates is analogous to that of FIG. 7, the rollers 31, 32 being guided parallel to one another. The plates 11, 12 are held decoupled from one another in a frame, the horizontal force measurement cells 21, 22 being arranged outside the plates 11, 12.

FIG. 9 is a schematic illustration of the projection of force vectors onto a person 1 and onto the background of said person 1. The person 1 stands on two force measurement plates 11, 12, and the respectively determined resulting force vectors F_{R}, F_{RL} for the right foot and left foot are projected onto the body of the person 1 and the background. Here, the resulting force vector F_{RR} represents the force vector which is calculated from the respective support points on the right force measurement plate 11, a known centroid height and a vertical force distribution on the two force measurement plates 11, 12 in relation to one another. The profile of the resulting force vector F_{RR} on the right, and the respective force introduction point are illustrated on the body of the person 1. The force is introduced into the right leg in the heel region, whereas the force is introduced into the left leg in the forefoot area so that the resulting force vector F_{RL} of the left leg is illustrated inclined with the vertical, and the resulting force vector F_{RL} projected onto the body passes through the forefoot area. A second form of representation is likewise illustrated in FIG. 9, where the resulting force vector F_{RL} of the left leg is illustrated on a background. For reasons of clarity, this is illustrated in a setback fashion such that the broken line is illustrated offset. To improve discernability, it is possible to illustrate the two resulting force vectors F_{RR}, F_{RL} with differing brightnesses or in different colors.

FIG. 10 illustrates a variant of the invention in the case of which the projected force vectors are guided through the body centroid 2. The illustration is analogous to the illustration in accordance with FIG. 9, either on the body of the person 1 or on a background; in this case, the body centroid height can be input manually, or can be determined for example via the detection of the horizontal forces in the frontal plane or sagittal plane.

FIG. 11 illustrates a possible method for calibrating a display device 3. The display device, for example in the form of a beamer, covers a measurement field 10 which is defined by its corner points. The two force measurement plates 11, 12 are arranged within the measurement field 4. The two force measurement plates 11, 12 are arranged next to one another and in a substantially rectangular fashion. Arranged at the free corner points of the measurement plates 11, 12 are sensors 41, 42, 43, 44 which can be designed as photosensitive sensors. Alternatively to this, markers can be arranged at the corner points of the measuring area, which is defined by the force measurement plates 11, 12. For the purpose of autocalibration, starting from a first lateral edge, a light strip or a darkened strip is moved to the opposite side over the measurement field 4. In the exemplary embodiment illustrated, the strip is firstly moved to the right from the left lateral edge, as indicated by the arrow. The display device 13 illuminates the measurement field 4 completely at first, and then the strip moved with different brightness firstly from the left lateral edge in the direction of the right lateral edge. Subsequently, a second strip is moved with different brightness from the upper lateral edge in the direction of the lower lateral edge of the measurement field 4. In this procedure, within the illuminated or dark area the strips consecutively sweep over the photosensitive sensors 41, 42, 43, 44 of the measurement plates 11, 12 such that it is possible to determine their position and size, given a known size of the measurement plates. An evaluation unit (not illustrated), generally a computer, therefore has information as to where the measurement plates 11, 12 are arranged in the measurement field 4 and concerning the distances from the display device 3 to the force measurement plates 11, 12. It is thereby possible to display the forces and resulting force vectors not only qualitatively, but also in a quantitative fashion. Such an illustration as known from FIG. 10 is shown in the illustration on the right in FIG. 11. Both the body centroid 2 and the resulting force vectors F_{RR}, F_{RL} can be illustrated on the basis of the values determined by the force measurement plates 11, 12, and projected onto the body of the person 1.

FIG. 12 illustrates a variant of the autocalibration in which the autocalibration is performed by a stepwise reduction of the illuminated area. The reduced areas are designated by roman numerals. The measurement field 4 is firstly reduced from the left until the left rear sensor 42 is reached, the result being the illuminated area I. Subsequently, said area is further reduced in a scaled fashion such that the areas II, III, IV, V result in sequence. The reduction is continued until, ideally, only the measurement point of the sensor 42 is illuminated, which is indicated by the field VI.

The method is then continued in the right illustration of FIG. 12, and the illuminated area of the area VII to XII is carried out in the respective steps. Said reduction of the illuminated areas is undertaken for all the sensors of the measurement plates 11, 12 such that all the position data of the sensors 41, 42, 43, 44 of the force measurement plates 11, 12 are known in a way similar to the method according to FIG. 11.

FIG. 13 illustrates a variant of the autocalibration. A pattern 400 in the form of a rectangle is projected into the measurement field 4 by the display device 3. Subsequently, the left lower point 401 of the pattern 400 is drawn onto the left lower sensor 41, which can also be designed as a marker. Subsequently, the left upper point 402 is drawn onto the left lower sensor 42 or marker, the same being done with the right upper point 403 of the pattern 400, which is drawn onto the rear right sensor 43. Subsequently, the right lower point of the pattern 400 is drawn onto the right front sensor 44 such that, starting from the known assignment of the sensors 41, 42, 43, 44 to one another and the positions of the corner points 401, 402, 403, 404 of the pattern 400, an autocalibration is carried out by adapting the pattern 400 to the sensor surface which is formed by the corner points of the force measurement plates 11, 12 at which the sensors 41, 42, 43, 44 are arranged.

FIG. 14 illustrates a variant of the configuration of the force measurement plate 11 in a plurality of views. In this case, the force measurement plate 11 is mounted on spherically shaped sensors 21, 22 which are supported in retainers 200. The retainers 200 are mounted on the force measuring plate 11 and a carrier plate 110 arranged underneath the latter. It is thereby possible for the spherically designed sensors 21, 22 to exert a roller bearing function, as a result of which the force measurement plate 11 is mounted on the carrier plate 110 in a fashion decoupled in the directions of the horizontal force. It is also possible in principle to arrange the retainers 200 either on the force measuring plate 11 or the carrier plate 110. The retainers 200 can permit a displacement in a direction of horizontal force when they are designed as rails. The retainers 200 serve the purpose in principle of ensuring a fundamental assignment of the force measurement plate 11 to the carrier plate 110. Should this come about in some other way, the retainers 200 can also be eliminated.

In order to prevent twisting of the force measurement plate 11 about the vertical axis in the event of application of torques, the force measurement plate 11 is mounted on a parallelogram guide 100 which respectively absorbs a horizontal force component. The force measurement plate 11 therefore cannot be twisted about a vertical axis in relation to the carrier plate 110.

In addition to the spherical structure of the sensors 21, 22, it is also possible to design the sensors 21, 22 to be only partially spherical, as is shown in the right, lower illustration. The sensors 21, 22 are designed in this case as partially spherical elements which have a cross section in the shape of a half moon. It is also possible in principle for the sensors 21, 22 to be of barrel-like or cylindrical configuration in order to enable a coupling in the horizontal force direction. In the case of such shaping, as well, it is possible to employ a design with an open cross section in order to be able to record vertical forces and other force components, for example via strain gauges. The configuration in a half-moon shape, that is to say the partially spherical configuration, with a hollow cross section can enable acquisition of the vertical forces, for example via the application of strain gauges or other known force transducers. Owing to the functions of integration of the roller bearing in the sensors 21, 22, there is no longer any need to record vertical forces separately underneath the force measurement plate 11 or the carrier plate 110. In addition, this enables separate functions and modules to be combined with one another in order to implement a reduced overall height. The sensors 21, 22 are therefore simultaneously bearings, force sensors and decoupling devices which enable displacement in at least one horizontal force direction.

1. A method for determining and displaying horizontal and vertical forces acting on a person while standing, the method comprising:
providing a display device and two separate, intercoupled force measurement plates, each force measurement plate being receptive of one foot of the person positioned thereon, a support point being determined between each foot and a respective force measurement plate;
calculating a resulting force vector from the respective support points of the two force measurement plates, from a known body centroid height and from a vertical force distribution of the two force measurement plates relative to one another;
displaying the force vectors with the aid of the display device.

2. The method as claimed in claim 1, wherein the vertical force component, with respect to the respective resulting support point is represented for each leg of the person.

3. The method as claimed in claim 1, wherein an overall centroid is determined from support point data of the force measurement plates, and the resulting force vector of the two force measurement plates is calculated and displayed.

4. The method as claimed in claim 1, wherein, before a resulting force vector is displayed, a plurality of sensors or markers at at least one force measurement plate are used for auto-calibration of display device relative to the at least one force measurement plate by scanning a measurement field in which the force measurement plates are located.

5. The method as claimed in claim 4, wherein the plurality of sensors include photosensitive sensors which are arranged at measurement points of at least one force measurement plate, and auto-calibration is carried out by illuminating the entire measurement field and subsequently reducing the illuminated measurement field and selectively activating photosensitive sensors.

6. The method as claimed in claim 4, wherein, for the purpose of auto-calibration, the display device projects patterns into the measurement field and adapts the patterns such that patterns correspond to sensors or markers at at least one of the force measurement plates.

7. The method as claimed in claim 4, wherein the auto-calibration is carried out by the display device as a function of patterns or coordinates in the measurement field.

8. The method as claimed in claim 1, wherein the resulting force vector is at least one of projected onto the person standing on the force measurement plates, and displayed in an image of the display device.

9. The method as claimed in claim 1, wherein the body centroid height is input manually or determined by detecting the horizontal force in the frontal plane or sagittal plane.

10. A device for carrying out a method as claimed in claim 1, the device comprising the two separate force measurement plates that are provided with sensors for recording at least one of forces and moments and are connected to an evaluation device via which the acquired measured variables are evaluated, wherein the force measurement plates are mounted decoupled in at least one horizontal force direction.

11. The device as claimed in claim 10, wherein two force measurement plates are mounted decoupled in different horizontal force directions.

12. The device as claimed in claim 10, wherein the sensors are arranged below or alongside the force measurement plates.

13. The device as claimed in claim 10, wherein the force measurement plates are mounted on rollers.

14. The device as claimed in claim 10, wherein the sensors are designed as rolling elements on which the force measurement plates are mounted.

15. A method for determining and displaying horizontal and vertical forces acting on a person while standing, the method comprising:
providing a display device and two force measurement plates, each force measurement plate being receptive of one foot of the person standing thereon, a support point being determined between each foot and a respective force measurement plate;
calculating a resulting force vector based on the support points, a known body centroid height, and a vertical force distribution of the force measurement plates relative to one another;
displaying the force vectors using the display device.

16. The method as claimed in claim 15, further comprising representing the vertical force component with respect to the respective resulting support point for each leg of the person.

17. The method as claimed in claim 15, further comprising:
determining an overall centroid from support point data of the force measurement plates;
calculating and displaying the force vector of the two force measurement plates.

18. The method as claimed in claim 15, wherein, before displaying a resulting force vector, auto-calibrating the display device relative to the at least one force measurement plate using a plurality of sensors or markers at at least one force measurement plate and scanning a measurement field in which the force measurement plates are located.

19. The method as claimed in claim 18, wherein the plurality of sensors include photosensitive sensors which are arranged at measurement points of at least one force measurement plate, and the auto-calibrating is carried out by illuminating the entire measurement field and subsequently reducing the illuminated measurement field and selectively activating photosensitive sensors.

20. The method as claimed in claim 18, wherein, for the purpose of the auto-calibrating, projecting patterns into the measurement field with the display device and adapting the patterns such that the patterns correspond to sensors or markers at at least one of the force measurement plates.
